(19) United States
(12) Patent Application Publication
(10) Pub. No.: US 2026/0345919 A1
(43) Pub. Date: Nov. 5, 2026

(54) Smallpox Vaccination Device

(71) Applicants: Retractable Technologies, Inc., Little Elm, TX (US); Thomas J. Shaw, Frisco, TX (US)

(72) Inventors: Thomas J. Shaw, Frisco, TX (US); Jenny Margolis, Allen, TX (US); Ni Zhu, McKinney, TX (US)

(21) Appl. No.: 19/744,510

(22) Filed: Jul. 16, 2026

Related U.S. Application Data
(63) Continuation of application No. 18/155,963, filed on Jan. 18, 2023.

(51) Int. Cl. A61M 5/32; A61M 5/50

(52) U.S. Cl. CPC A61M 5/3243; A61M 5/3286; A61M 5/50

(57) ABSTRACT

Various embodiments of a safe, inexpensive and disposable medical device useful for administering vaccinations are generally characterized by a needle having a bifurcated needle tip with a solid core that is attached in fixed relation to a needle holder, dipped in or coated with a vaccine or other similar medicament, and reciprocated multiple times with or without a tubular barrel and a compression spring to effectuate sufficient intradermal penetration to transfer a medically effective dose to an identified skin area of a recipient. An active or passive safety feature operable to guard the bifurcated needle tip against accidental contact with another person or object is also disclosed.

### CROSS-REFERENCE TO RELATED APPLICATIONS

This application is a continuation of U.S. Application No. 18/155,963, filed on Jan. 18, 2023, by Thomas J. Shaw et al. and entitled “Smallpox Vaccination Device,” the disclosure of which is hereby incorporated by reference in its entirety for all purposes.

### FIELD OF THE INVENTION

This invention relates to an inexpensive, safe and disposable device or apparatus useful for efficiently and reliably administering a vaccine to an individual. The subject device is suitable for use in administering smallpox vaccine but is also believed to be satisfactory for use in administering other vaccines where multiple, relatively shallow skin pricks, intradermal, transdermal or subcutaneous injections or penetrations are medically preferred and recommended over intramuscular injection. In its various embodiments, the subject device is designed for one-time use, is desirably made of medical-grade plastic with a metal needle rod having a solid core and a bifurcated needle tip. The subject device is desirably rendered “safe” following administration of a vaccine to a single patient either by withdrawing the needle back inside a protective housing or by advancing a protective needle guard to shield the bifurcated needle tip against subsequent accidental needle stick injuries prior to disposal.

### BACKGROUND

Each vaccine has a recommended administration route and site that are included in the manufacturer’s package insert for each vial of vaccine. Deviation from the recommended route can reduce vaccine efficacy or increase local adverse reactions. Bifurcated needles (solid rods with two prongs on the front) are well known for use in administering single doses of a vaccine to a patient and, more particularly, for holding a medically effective dose of reconstituted, freeze-dried smallpox vaccine on and between the two prongs of a bifurcated needle during the vaccination process. The amount of vaccine held by a bifurcated needle during a single insertion or dipping of a bifurcated needle into a vial is typically sufficient to deliver a full dose of vaccine to a recipient following withdrawal of the needle from the vial. The vaccine is desirably delivered to the recipient by repeatedly advancing the bifurcated needle tip against the skin surface and into the skin in a series of separate contact events (up to 15 or more) during which most if not all the vaccine dose is transferred from the bifurcated needle tip to a grouping of skin pricks, punctures and shallow penetrations disposed within a small circular area defining the vaccination site.

Each full vial desirably contains enough vaccine for a plurality of individual doses, sometimes as many as 100 doses per vial. However, once a bifurcated needle tip has been introduced into a vaccine vial, has been withdrawn from the vial and has subsequently contacted the skin of a patient, any subsequent insertion of a needle into the vial should be done using a different needle to avoid contaminating any unadministered vaccine remaining inside the vial. Where an elastomeric membrane or other cover remains in place over an open end of the vial, reuse of a contaminated needle is undesirable to avoid contaminating the unused vaccine and to avoid dulling the needle from multiple membrane penetrations, thereby reducing effectiveness for skin penetration and vaccine delivery.

Although the use of solid-core needles with bifurcated needle tips has previously provided advantages over other prior art devices to healthcare workers and recipients in the administration of vaccines, further technological advances and improvements are needed that will enable better utilization of needles and will provide additional protective features and safety benefits.

### SUMMARY OF THE INVENTION

Various embodiments of a safe, inexpensive and disposable medical device for use in administering vaccinations are disclosed. The subject embodiments are generally characterized by a needle having a bifurcated needle tip with a solid core that is attached in fixed relation to a reciprocating needle holder. The subject medical device is desirably configured to allow the bifurcated needle tip to be dipped into or coated with a vaccine or other medicinal fluid, and then advanced into contact with a patient’s skin with sufficient force to prick, abrade or penetrate the epidermis. The bifurcated needle tip can be coated with vaccine by dipping or inserting the needle into a conventional medicine vial or other open reservoir of the vaccine or medicament.

Use of a bifurcated needle tip is preferred because it provides an increased surface area and supports a larger volume of the vaccine or other medicinal fluid on the needle and also provides a greater surface area than a single needle for contacting, pricking, puncturing or abrading the skin surface to promote transfer of the vaccine onto or into the skin. Following initial contact with the skin, the bifurcated needle tip is preferably withdrawn from contact with the patient’s skin and then reciprocated for a plurality of cycles (up to 15 or more, for example) to promote transfer of the vaccine onto and into the skin sufficiently to achieve the desired therapeutic effectiveness. In some embodiments of the invention, a coil spring is provided in the subject medical device to facilitate withdrawing the bifurcated needle tip and then reciprocating the needle tip back into contact with the skin for a plurality of cycles.

The medical device of the invention also desirably comprises at least one safety feature that can be actively or passively implemented to prevent accidental needle sticks from occurring following use of the device for its principal intended purpose of administering vaccines or similar medicinal fluids. The safety feature can embody any of several similarly effective structural elements such as, for example, a sliding, rotatable or pivotable protective guard or latch, a selectively positionable, frictionally engageable retainer member, or the like.

In general, the present invention is intended for use in administering intradermal and transdermal vaccines where a medically effective amount of the vaccine is supported between the two prongs of a solid-core, bifurcated needle that is used to transfer of the vaccine into the skin without inserting the needle to a subcutaneous depth.

Other benefits and advantages of the subject medical devices will likewise become more apparent to those of ordinary skill in the art upon reading this disclosure in relation to the accompanying drawings.

### BRIEF DESCRIPTION OF THE DRAWINGS

The apparatus of the invention is further described and explained in relation to the following drawings wherein:

FIG. 1 is a front perspective view of an embodiment of the medical device of the invention with a bifurcated needle tip projecting forwardly from the barrel;

FIG. 2 is an exploded front perspective view of the medical device of FIG. 1 rotated 90 degrees around its longitudinal axis;

FIG. 3 is a top plan view of the medical device of FIG. 2 with the bifurcated needle tip retracted into the barrel;

FIG. 4 is a cross-sectional view taken along line 4—4 of FIG. 3;

FIG. 5 is a top plan view of the medical device of FIG. 3 with the plunger fully advanced, causing the bifurcated needle tip to project forwardly from the barrel;

FIG. 6 is a cross-sectional view taken along line 6—6 of FIG. 5;

FIG. 7 is a front perspective view of another embodiment of the medical device of the invention with a bifurcated needle tip projecting forwardly from the barrel;

FIG. 8 is an exploded front perspective view of the medical device of FIG. 7;

FIG. 9 is a front elevation view of the medical device of FIG. with the slidable needle shield moved forwardly from the position shown in FIG. 7 to cover the bifurcated needle tip and protect against accidental needle stick injuries;

FIG. 10 is a cross-sectional view taken along line 10—10 of FIG. 9; FIG. 11 is an enlarged detail view taken from FIG. 10;

FIG. 12 is a side elevation view of the medical device of FIG. 7;

FIG. 13 is a cross sectional view taken along line 13—13 of FIG. 12; and FIG. 14 is an enlarged detail view taken from FIG. 13;

FIG. 15 is a front perspective view of another embodiment of the medical device of the invention with the slidable needle shield advanced to cover the bifurcated needle tip prior to and after use of the device to administer a vaccine;

FIG. 16 is an exploded front perspective view of the medical device of FIG. 15;

FIG. 17 is a top plan view of the medical device of FIG. 15 in which the bifurcated needle tip is in a protected position inside the needle shield;

FIG. 18 is a cross-sectional view taken along line 18—18 of FIG. 17;

FIG. 19 is an enlarged detail cross-sectional view taken from the distal end portion of FIG. 18;

FIG. 20 is a cross-sectional view of the medical device of FIG. 15 as seen in FIG. 18 except that the needle holder is fully depressed inside the barrel, the spring is fully compressed, and the bifurcated needle tip projects forwardly from the open end of the needle shield;

FIG. 21 is an enlarged detail cross-sectional view taken from the distal end portion of FIG. 20;

FIG. 22 is an exploded front perspective view of another embodiment of the medical device of the invention that is configured similarly to the medical device of claim 16 except that the handle and thumb cap are detachable from the proximal end of the needle holder;

FIGS. 23-25 are substantially similar to FIGS. 17-18, 20, respectively, except that the needle holder embodies the detachable handle of FIG. 22;

FIG. 26 is a front perspective view of another embodiment of the medical device of the invention that comprises a selectively pivotable, elongate needle guard to protect the bifurcated needle tip against accidental needle sticks prior to and after use of the device;

FIG. 27 is a left side elevation view of the medical device of FIG. 26;

FIG. 28 is an exploded view of the medical device of FIG. 26 showing the elongate needle guard pivoted forwardly relative to the barrel;

FIG. 29 is a top plan view of the medical device of FIG. 28 when fully assembled, with the elongate needle guard pivoted forwardly to cover the bifurcated needle tip;

FIG. 30 is a front perspective view of another embodiment of the medical device of the invention that is similar to the embodiments of FIGS. 17, 23 but having a different needle holder and thumb cap configuration, with the bifurcated needle tip protected inside a circular needle guard when not in use;

FIG. 31 is an exploded view of the medical device of FIG. 30;

FIG. 32 is a left side elevation view of the medical device of FIG. 30;

FIG. 33 is a cross-sectional view taken along line 33---33 of FIG. 32 showing a partially relaxed compression spring biasing the needle holder rearwardly to maintain the tip end of the bifurcated needle inside the circular needle guard;

FIG. 34 is an enlarged detail view of the distal end portion of FIG. 33; and FIG. 35 is a cross-sectional view as in FIG. 33 with the needle holder fully depressed inside the barrel, thereby compressing the spring and causing the bifurcated needle tip to project forwardly of the circular needle guard.

### DESCRIPTION OF PREFERRED EMBODIMENTS

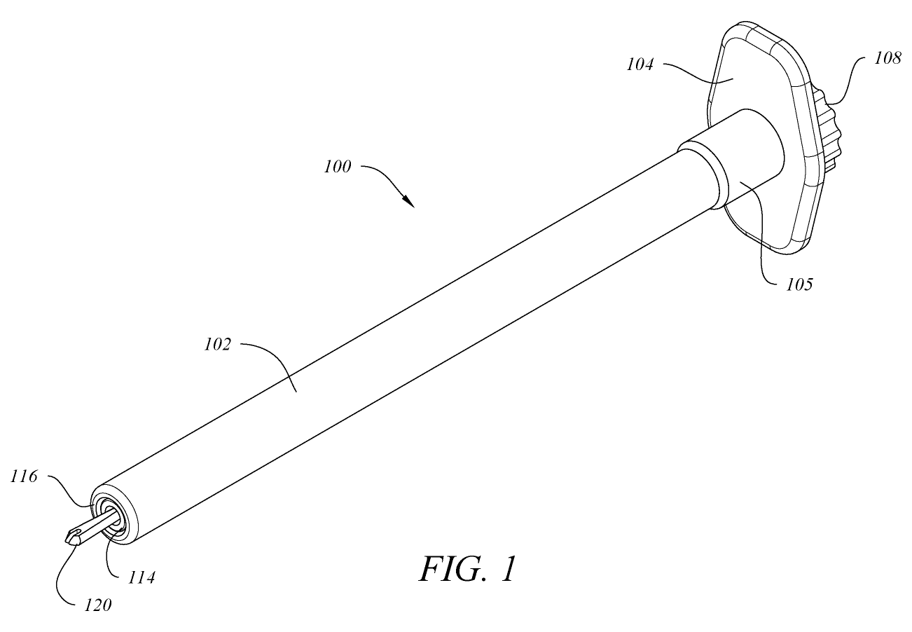

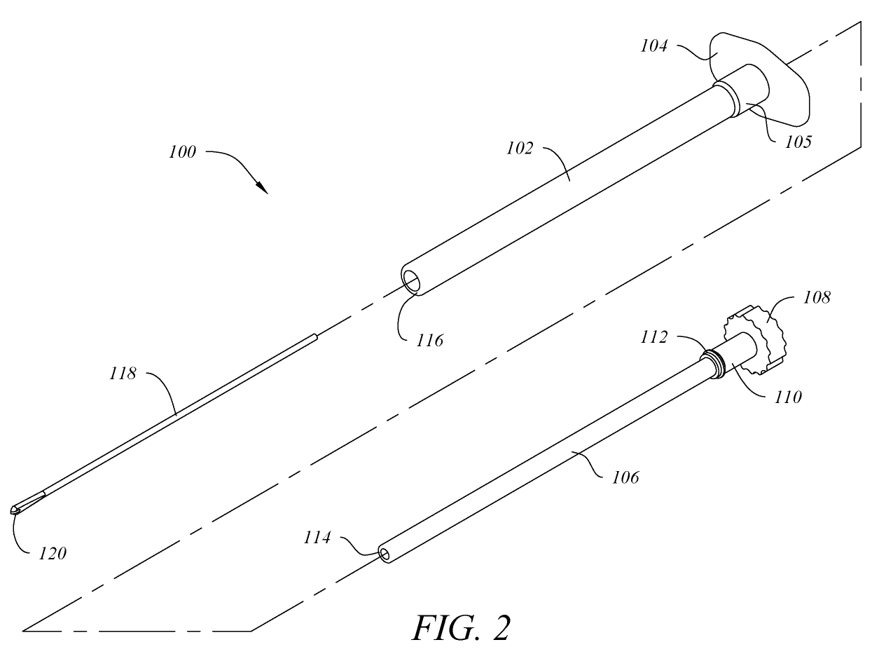

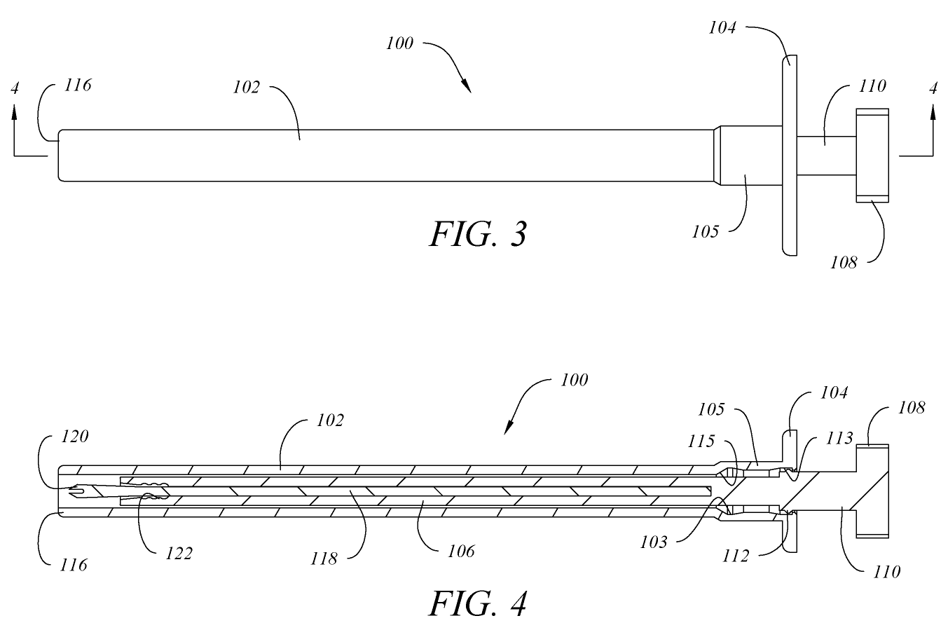

Referring to an embodiment of the invention as disclosed in FIGS. 1-2, medical device 100 comprises tubular barrel 102 having a front opening 116 and a rear opening defined by a radially transitioning retainer collar 105 (further explained in relation to FIGS. 3-4 below) and a transversely projecting flange member 104. Needle holder 106 is desirably insertable forwardly through flange member 104 and retainer collar 105 of coaxially aligned tubular barrel 102. Needle holder 106 desirably further comprises a front opening 114 that communicates with a centrally disposed, forwardly facing internal bore section having a length and inside diameter sufficient to receive needle shaft 118 into fixed longitudinal and rotational engagement with needle holder 106. During assembly of medical device 100, needle holder 106 can be inserted forwardly through flange 104 and retainer collar 105 of barrel 102. Following insertion of needle holder 106 into barrel 102, needle shaft 118 can be inserted through front opening 116 of barrel 102, through front opening 114 of needle holder 106 and into seated engagement inside the longitudinal bore of coaxially aligned needle holder 106, at which time the rear portion of bifurcated needle tip 120 can be cemented or otherwise attached to the inwardly tapered and undulating inside wall 122 of needle holder 106 as seen in FIGS. 4 and 6. Alternatively, in a modification of FIG. 2, the proximal end of needle shaft 118 can be inserted through front opening 114 and into seated engagement inside needle holder 106, after which the proximal end of bifurcated needle tip 120 can be cemented or otherwise attached inside needle holder 106 prior to inserting the distal end portion of needle holder 106 into the proximal end of tubular barrel 102.

Although all embodiments of the invention are depicted without packaging, it will be appreciated by those skilled in the art upon reading this disclosure that all embodiments are desirably packaged and sterilized prior to use in accordance with applicable regulations for such medical products. FIGS. 3-4 depict medical device 100 in the “safe” position as it could be manufactured and packaged for transportation and storage prior to use. Referring to FIGS. 3-4, medical device 100 is assembled with needle shaft 118 seated and attached inside needle holder 106, with bifurcated needle tip 120 projecting forwardly from needle holder 106 but still disposed inside the tubular bore of barrel 102 and rearwardly of front opening 116 of barrel 102 to avoid accidental dulling of the needle tip or accidental needle sticks prior to use. Needle holder 106 is maintained in this position relative to barrel 102 by frictional engagement between annular ring 112 disposed inside retainer collar 105 and a first cooperatively configured annular recess 113 disposed inside flange 104. The axial position of needle holder 106 inside barrel 102 is selectively controlled by applying forwardly directed pressure to control knob 108 while holding barrel 102 by flange 104.

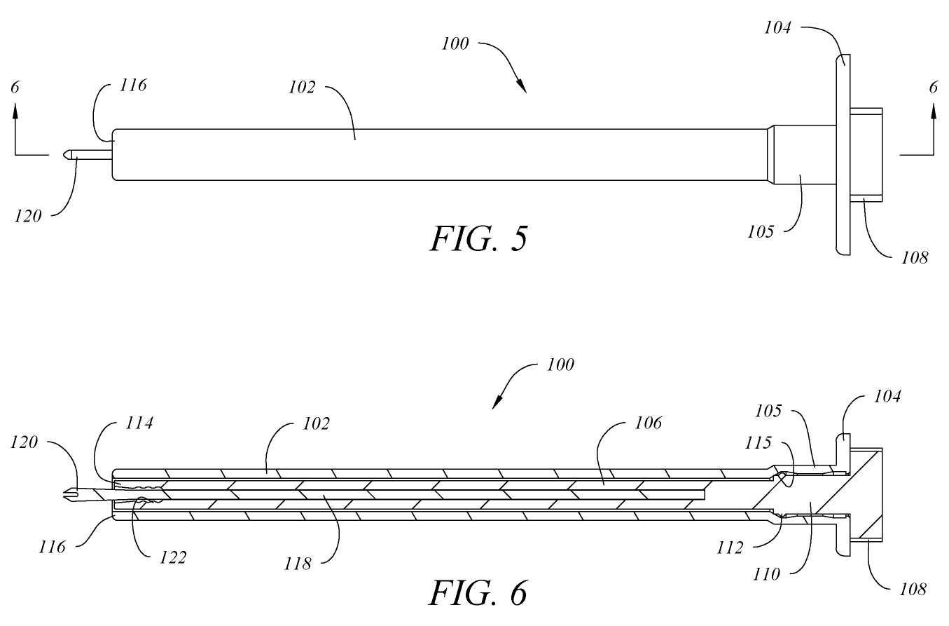

FIGS. 3 and 4 depict needle holder 106 selectively positioned to maintain separation between control knob 108 and flange 104 along handle section 110 prior to use of medical device 100. FIGS. 5-6 depict control knob 108 and handle section 110 advanced forwardly to a use position where the distal face of control knob 108 abuts the proximal face of flange 104. In the use position shown in FIGS. 5 and 6, annular ring 112 is advanced to a second cooperatively configured annular recess 103 disposed at the front shoulder 115 of radially transitioning retainer collar 105, and bifurcated needle tip 120 projects forwardly from front opening 116 of barrel 102. With bifurcated needle tip 120 disposed in the position shown in FIGS. 5-6, it can be dipped into a reservoir (not shown) of vaccine or other medicinal fluid and then contacted the skin of an individual receiving the vaccination. In this embodiment of medical device 100, the maximum penetration depth of bifurcated needle tip 120 into or through the epidermis of the recipient is determined by how far forward the bifurcated needle tip 120 extends forwardly of front opening 116 of barrel 102, which is in turn controlled by two factors: (a) the axial distance that bifurcated needle tip 120 is recessed inside front opening 116 of barrel 102 when medical device 100 is in the pre-use position of FIGS. 3-4; and (b) the axial distance between the position of annular ring 112 when needle holder 106 is disposed as shown in FIG. 4 and the position of annular ring 112 when needle holder 106 is disposed as shown in FIG. 6.

Medical device 100 can be used to administer a vaccine or other medicament by a single contact or insertion of bifurcated needle tip 120 with or into the skin of the recipient or can be manually reciprocated relative to the skin of the patient for multiple pricks or sticks while holding barrel 102 of medical device 100 in one hand similarly to how the user would hold a pencil while bifurcated needle tip 120 is projecting forwardly as positioned in FIGS. 5-6. Whether medical device 100 is used to administer one or several pricks to the skin of a patient, following the last contact with the patient’s skin, needle holder 106 and bifurcated needle tip 120 are desirably returned to the position shown in FIG. 4 by gripping control knob 108 and withdrawing needle holder 106 relative to barrel 102 until annular ring 112 releases from annular recess 103 (FIG. 6) and again frictionally engages the cooperatively configured annular recess 113 (FIG. 4) disposed in the opening through flange 104. Following completion of the procedure as described above, medical device 100 should not be used to treat another individual and should be deposited for responsible disposal into an approved “sharps” container with the bifurcated needle tip 120 still in the protected “safe” position as shown in FIGS. 3-4.

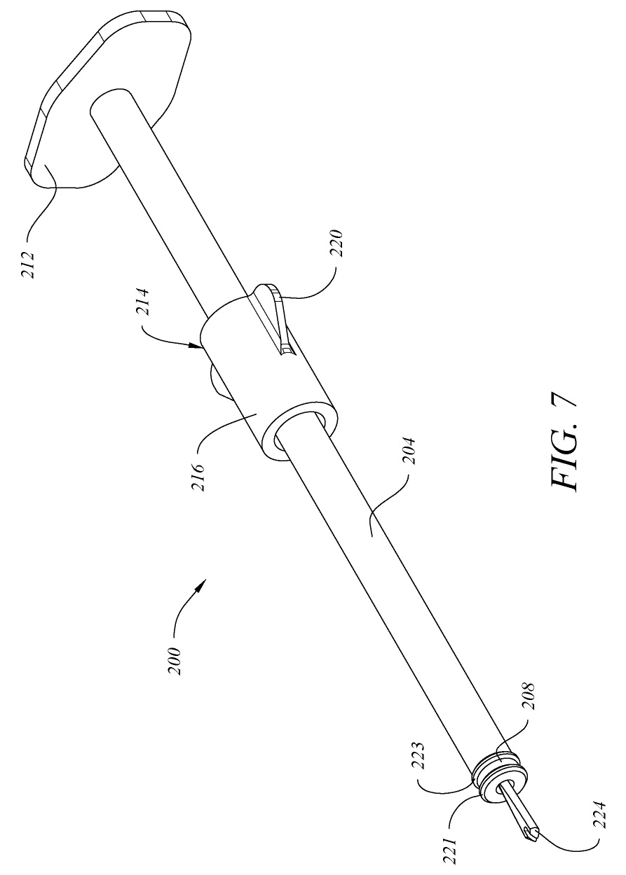

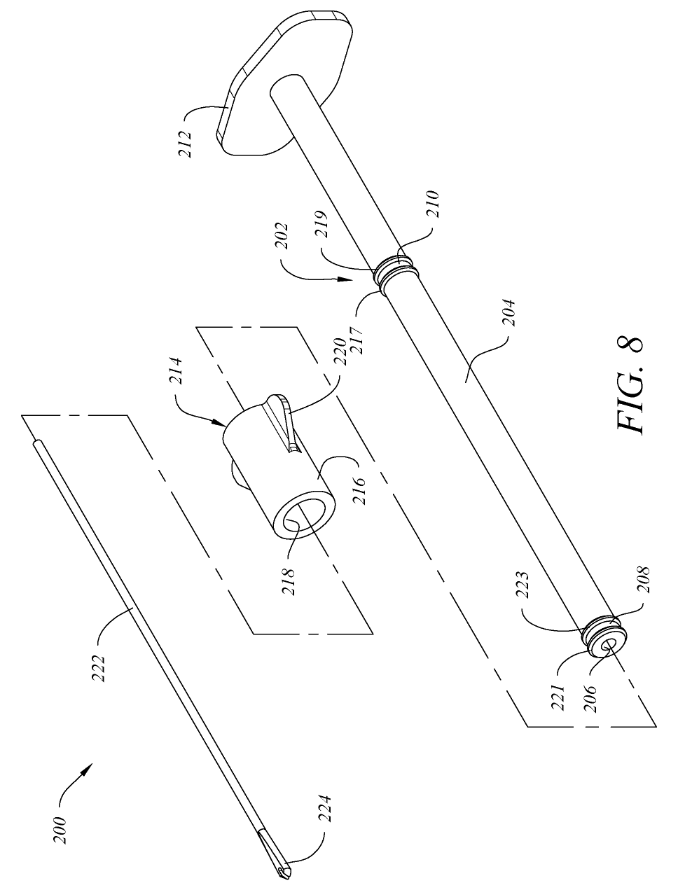

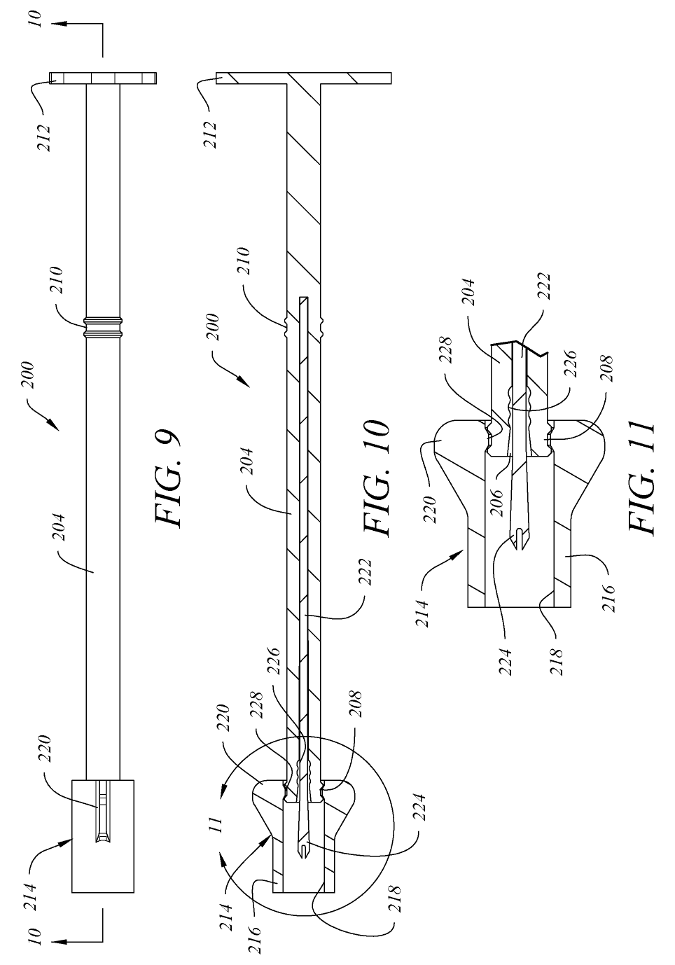

Another embodiment of the subject invention is further described and disclosed in relation to FIGS. 7-14 of the drawings. Referring to FIGS. 7-8, medical device 200 comprises needle holder 202 having an elongate tubular section 204 with a front opening 206 communicating with a centrally disposed axial bore into which a solid needle shaft 222 is insertable and attachable in fixed relation by introducing cement into a transition section with undulations 226 adjacent to front opening 206 or through use of another similarly effective attachment method, structure or composition. A forwardly projecting, bifurcated needle tip 224 made of steel or another similarly effective metallic or ceramic material is desirably coaxially aligned with and integrally connected to needle shaft 222. Either before or after inserting and seating needle shaft 222 inside needle holder 202 during the assembly of medical device 200, sliding safety member 214 is desirably installed over the forwardly facing distal end of needle holder 202. Sliding safety member 214 further comprises cylindrical body 216 with substantially cylindrical bore 218 and laterally projecting wings 220. Sliding safety member 214 is a selectively positionable safety device used to shield the forwardly projecting bifurcated needle tip 224 (visible in the use position shown in FIGS. 7 and 12-14) from accidentally sticking a patient or healthcare worker when medical device 200 is disposed in either the pre-use or post-use position (visible in FIGS. 9-11).

More particularly, sliding safety member 214 and needle holder 202 are desirably cooperatively configured to permit sliding safety member 214 to be moved selectively by a user from a “pre-use” position in which sliding safety member 214 is positioned around and in front of bifurcated needle tip 224 to protectively shield bifurcated needle tip 224 from incidental or external contact, to a “use” position in which sliding safety member 214 is retracted behind bifurcated needle tip 224 to expose the needle tip for use in administering a vaccination, to a “post-use” position in which bifurcated needle tip 224 is again advanced to cover, surround and shield the contaminated needle tip 224 during post-use handling and disposal. Sliding safety member 214 is desirably configured to frictionally and releasably engage needle holder 202 when disposed in the pre-use, use and post-use positions and to slide easily between the two positions when manually repositioning sliding safety member 214 as described above.

Referring to FIGS. 8, 10-11 and 13, in this embodiment of the invention, sliding safety member 214 desirably comprises a cylindrical inside surface having an inside diameter slightly greater than the outside diameter of annular shoulders 217, 219, 221, 223 to allow sliding safety member 214 to move past annular shoulders 221, 223 and travel unimpeded between them. Referring to FIGS. 10-11, 13, near the proximal end of sliding safety member 214, an inwardly facing annular rib 228 is desirably provided on inside wall 218. Annular rib 228 is cooperatively sized and configured to allow sliding safety member 214 to be pressed onto needle holder 202 and past annular shoulder 221 and into frictional engagement with annular seating groove 208 between annular shoulders 221, 223, thereby releasably holding sliding safety member 214 in its pre-use position, protecting bifurcated needle tip 224 during packaging, sterilization, shipping and storage. Following removal of packaging prior to use of medical device 200, sliding safety member 214 is desirably moved rearwardly past annular shoulder 223, across tubular shaft portion 204, past annular shoulder 217 and into frictional engagement with annular seating groove 210, leaving bifurcated needle tip 224 in an exposed, forwardly facing position. Repositioning of sliding safety member 214 is facilitated by applying rearwardly directed manual pressure to sliding safety member 214 using laterally projecting wings 220 against a resisting force applied manually to the rearwardly facing surface of transverse barrel flange 212.

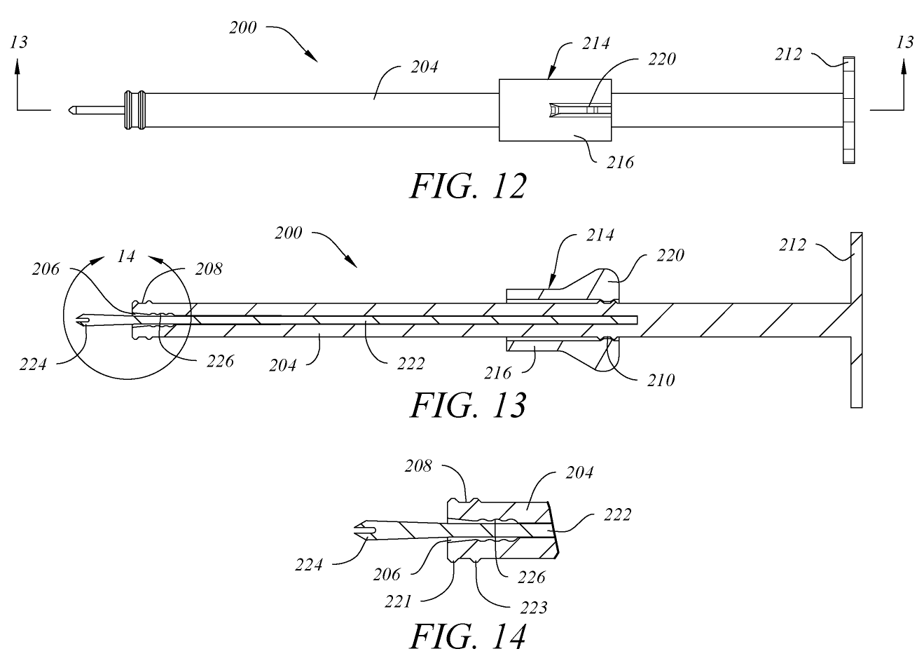

Medical device 200 can be used to administer a vaccine or other medicament by dipping bifurcated needle tip 224 into a reservoir containing the vaccine a single contact or insertion of bifurcated needle tip 224 with or into the skin of the recipient and then applied by manually reciprocated relative to the skin of the patient for multiple pricks or sticks while holding needle holder 204 of medical device 200 in one hand similarly to how the user would hold a pencil while bifurcated needle tip 224 is projecting forwardly as positioned in FIGS. 12-14. Whether medical device 200 is used to administer one or preferably a plurality (desirably up to about 15, for example) of pricks to the skin of a patient, following the last contact with the patient’s skin, slide member 214 is desirably returned to the position shown in FIGS. 9-11 by pushing it forwardly with sufficient force to overpressure the resistance provided by annular shoulder 217 until slide member 214 moves forward relative to needle holder 204 and inwardly facing annular rib 228 again frictionally engages the cooperatively configured annular groove 208 (FIGS. 10-11) disposed between annular shoulders 221, 223 to cover bifurcated needle tip 224 and protect users from accidental needle sticks. Following completion of the procedure as described above, medical device 200 should not be used to treat another individual and should be deposited for responsible disposal into an approved “sharps” container with bifurcated needle tip 224 still in the protected “safe” position as shown in FIGS. 9-11.

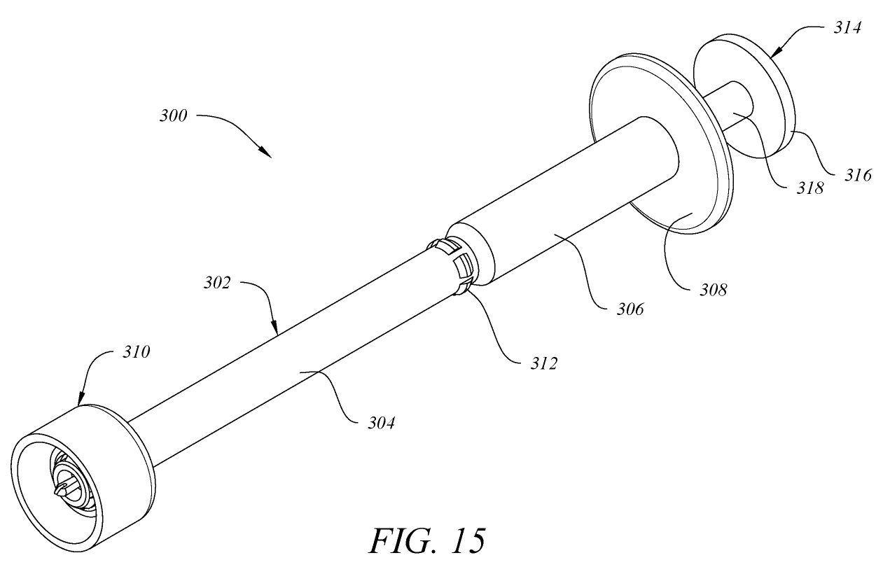

Another embodiment of the invention is disclosed in relation to FIGS. 15-21. Medical device 300 is desirably configured and used to administer vaccines similarly to the apparatus and method disclosed above in relation to the previous embodiments but differs from those embodiments at least with the inclusion of a compression spring that facilitates reciprocation of the needle holder and bifurcated needle tip relative to the barrel. Medical device 300 also differs from the previously disclosed embodiments with the provision of a circular needle guard that can support the device against the skin of the patient during use; can be configured to limit the dermal penetration of the bifurcated needle tip during a vaccination; and can be selectively moved to and releasably seated in an alternate position disposed rearwardly on the barrel to facilitate insertion of the bifurcated needle tip into a medicine vial prior to administering a vaccination.

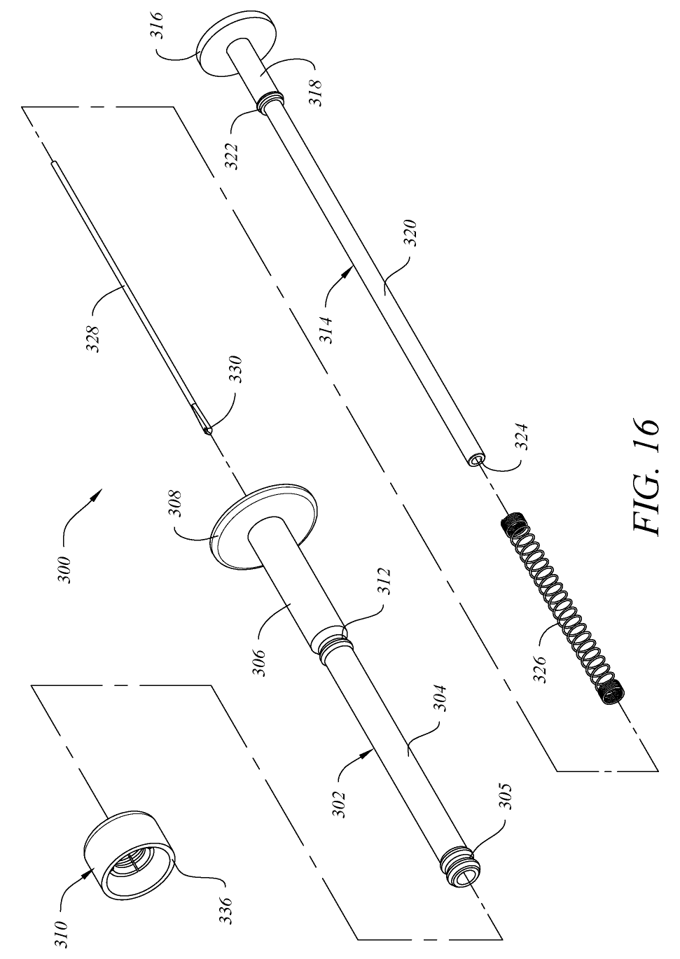

Referring to FIGS. 15-16, medical device 300 comprises tubular barrel 302, circular needle guard 310, needle rod 328 with bifurcated needle tip 330, needle holder 314 and compression spring 326. Tubular barrel 302 further comprises front section 304, rear section 306, front annular seat 305, rear annular seat 312 and transverse flange 308. Needle holder 314 further comprises elongate, generally cylindrical section 320 having front opening 324 and an internal bore cooperatively sized and configured to receive needle rod 328 in fixed relation to needle holder 314. At the rear of needle holder 314, the outside diameter of elongate, generally cylindrical section 320 desirably allows it to be inserted through uncompressed spring 326 prior to inserting tubular section 320 and spring 326 through a rearwardly facing opening in transverse flange 308 of barrel 302.

Following insertion of uncompressed spring 326 into the annulus disposed between the inside wall of rear section 306 and the outside wall of elongate, generally cylindrical section 320 that extends longitudinally between rear annular seat 312 of barrel 302 and rear annular seat 322 disposed forwardly of handle 318, handle 318 and transverse circular thumb cap 316 at the proximal end of needle holder 314 are releasably held in relation to barrel 302 by the frictional engagement between inwardly facing annular rib 338 disposed on the inside surface of rear section 306 and the outwardly facing annular seat 322 of needle holder 314.

During assembly of medical device 300, needle rod 328 can be inserted into fixed engagement with needle holder 314 either before or after needle holder 314 is inserted into the rearwardly facing opening of barrel 302. Once inserted into needle holder 314, needle rod 328 can be cemented or otherwise similarly affixed by known methods using the annular undulations disposed inside front opening 324 of needle holder 314.

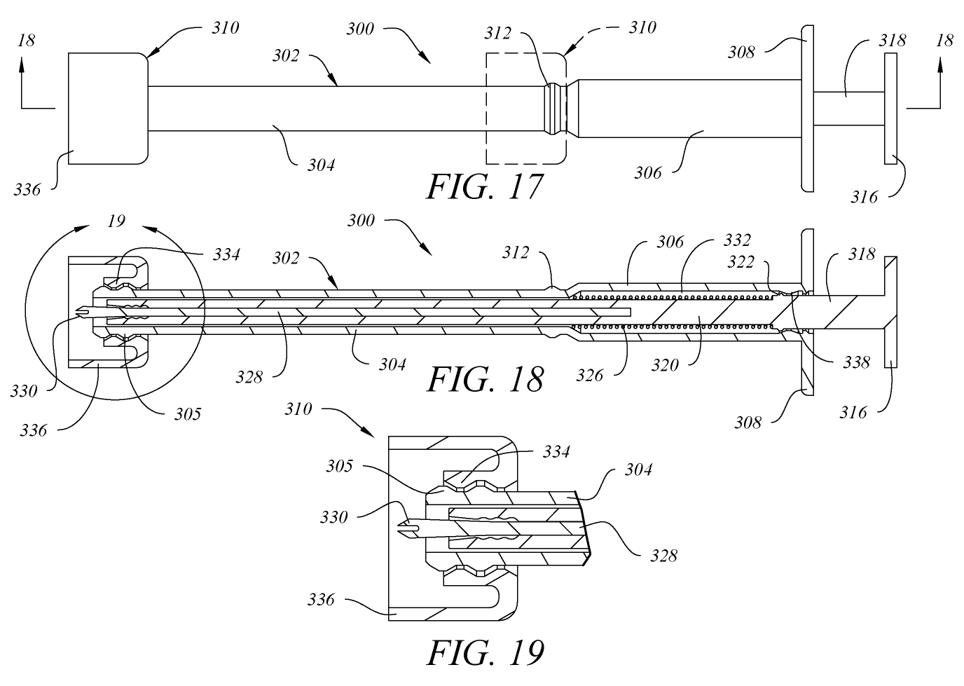

Referring to FIGS. 15, 17-19, circular needle guard 310 is desirably positioned as shown during assembly and, following packaging and sterilization, during shipment, and storage prior to use. In this embodiment, circular needle guard 310 comprises front opening 336 and a rear opening with a forwardly facing, generally cylindrical collar 334 that is cooperatively sized and configured to frictionally engage front annular seat 305 at front section 304 of tubular barrel 302. When medical device 300 is removed from its packaging and is being prepared for use in administering a vaccine, circular needle guard 310 can be selectively repositioned to the alternative position depicted in dashed outline in FIG. 17 to facilitate insertion of bifurcated needle tip 330 into a vial for dipping and/or coating of bifurcated needle tip 330 with a vaccine or other medicament prior to administering the dose to a patient or recipient. Once the dose of vaccine has been loaded onto bifurcated needle tip 330, circular needle guard 310 can again be moved to the first position (shown in solid outline in FIGS. 17-19) prior to contacting the skin of the recipient.

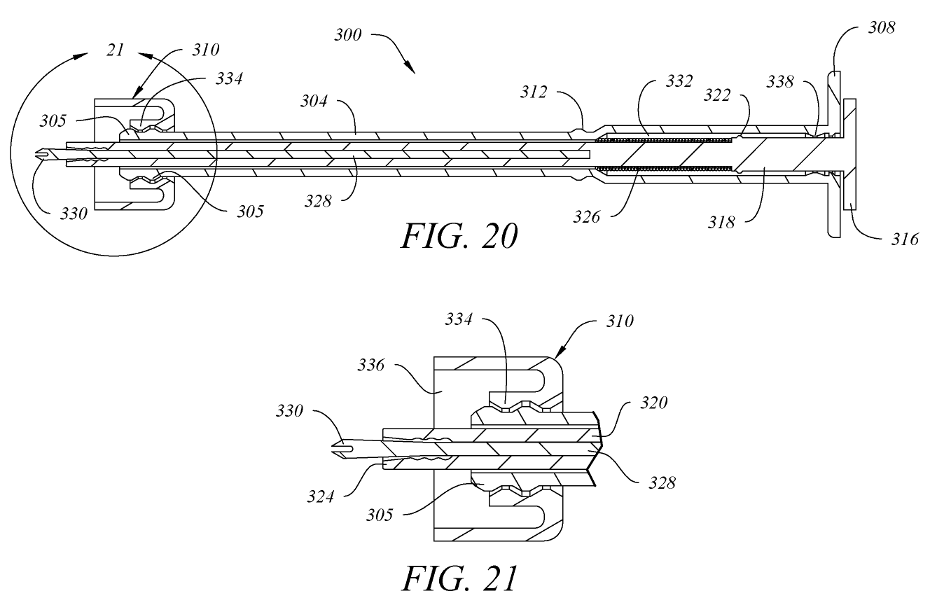

Using medical device 300 with reference to FIGS. 20-21 a vaccination is desirably administered to a recipient by pressing the circular front opening 336 of circular needle guard 310 lightly against the skin of the recipient and then sequentially pressing transverse circular thumb cap 316 of needle holder 314 forwardly against fingertip resistance applied to the front fact of transverse flange 308 of barrel 302 to overpressure the frictional holding force exerted by annular ridge 338 and move needle holder 314 forwardly relative to barrel 302, at the same time compressing spring 326 and causing bifurcated needle tip 330 to move forwardly to a more exposed position relative to circular needle guard 310 to initiate contact and commence transfer of the vaccine or other medicament to the skin of the recipient. In accordance with the drug manufacturer’s instructions provided with the vaccine, needle holder 314 and bifurcated needle tip 330 are then desirably reciprocated relative to the barrel 302 and circular needle guard 310 a prescribed number of times while holding the circular needle guard 310 in the same general location on the surface of the skin to maintain a vaccination situs within a circle having a diameter of generally prescribed dimensions such as, for example, from about 0.5 cm to about 2.5 cm.

Following application of the vaccine to the recipient in a dose and according to a regimen as recommended, compression spring 326 will relax to its uncompressed state and bifurcated needle tip 330 will return to a resting position inside circular needle guard 310, after which medical device 300 can be deposited into a sharps container for safe disposal as previously discussed.

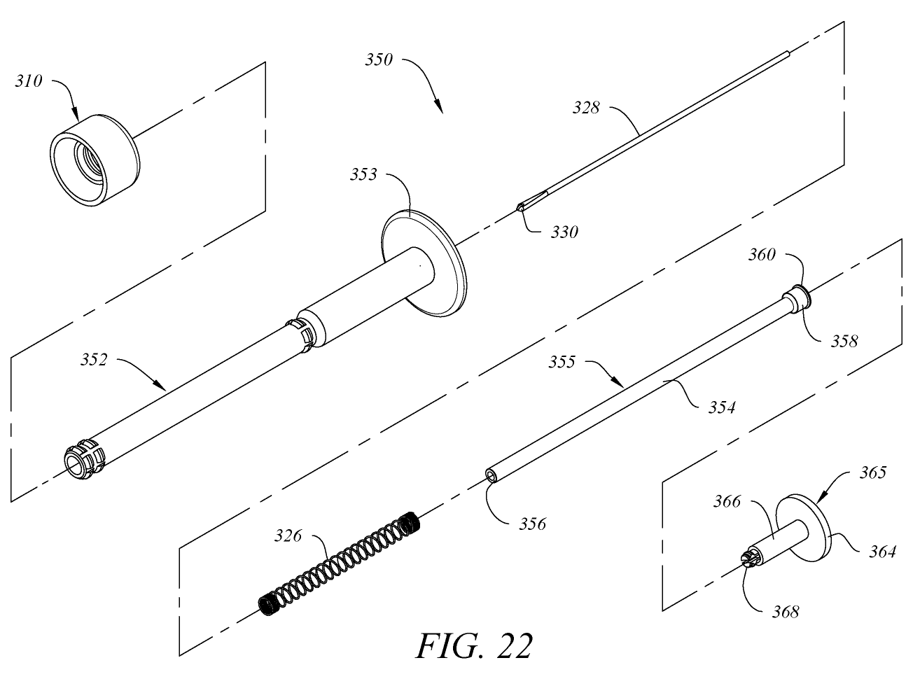

Referring to FIGS. 22-25, another embodiment of the invention is disclosed in relation to medical device 350, which embodies many structural elements the same or similar to some of those embodied in medical device 300, as discussed above. Medical device 350 comprises tubular barrel 352 with transverse barrel flange 353, circular needle guard 310, needle rod 328 with bifurcated needle tip 330 and compression spring 326. Needle holder 355 differs from needle holder 314 of medical device 300 in that it embodies a socket receptacle 358 sized and configured to receive a detachable handle 365 comprising socket plug 368 (insertable into the proximal end of socket receptacle 358), handle shaft 366 and circular thumb cap 364. Referring to FIGS. 22 and 24-25, socket receptacle 368 of needle holder 355 desirably comprises an outwardly flared, circumferentially extending lip 360 that reinforces the proximal end of needle holder 355 and abuts against inwardly projecting annular rib 360 disposed inside a cooperatively configured opening in the proximal end of transverse barrel flange 353 when compression spring 326 is in its relaxed position (shown in FIG. 24).

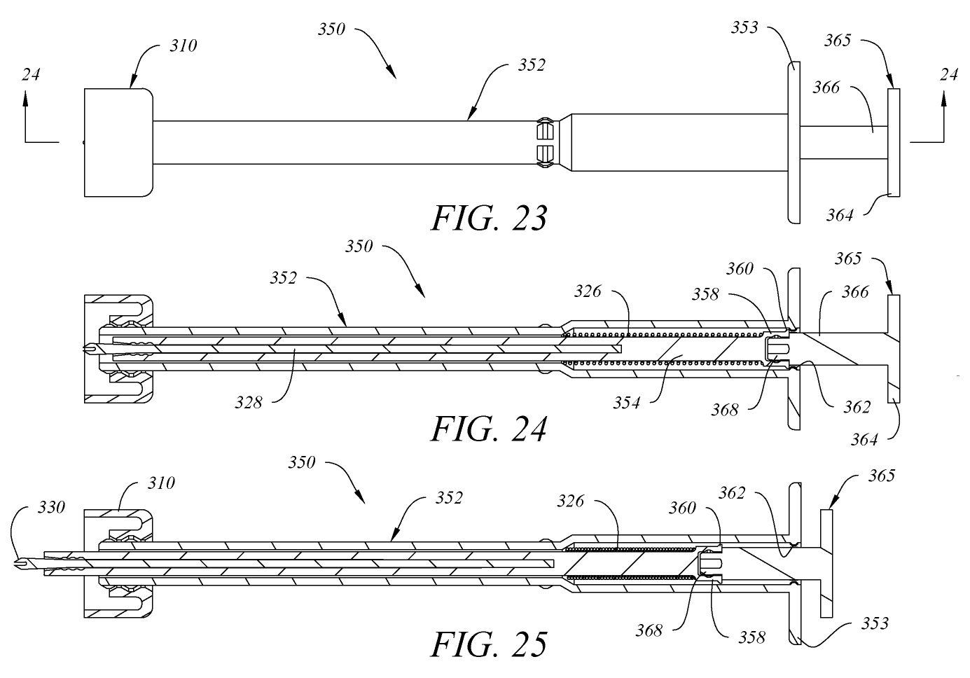

To administer a vaccine using medical device 350, after bifurcated needle tip 330 has been dipped in or coated with vaccine or another medicament, needle holder 355 is advanced forwardly from the position shown in FIGS. 23-24 to the position shown in FIG. 25, thereby compressing spring 326 and causing bifurcated needle tip 330 to project forwardly from circular needle guard 310 to contact the skin of the vaccine recipient. Following a protocol as previously described in relation to the prior embodiment, needle holder 355 can then be reciprocated relative to tubular barrel 352 and the skin of the recipient up to about 15 times to transfer the full dose of vaccine from bifurcated needle tip 330 to a skin area preferably disposed inwardly of the circumferential boundary of circular needle guard 310. During reciprocation of needle holder 355, compression spring 326 alternately biases needle holder 355 rearwardly to the position shown in FIGS. 23-24 before being advanced forwardly again by exerting manual pressure against the proximal surface of circular thumb cap 364 to compress spring 326 while applying a resistive finger force to the distally facing annular surface of transverse barrel flange 353. With each forward movement of needle holder 355 relative to tubular barrel 352, bifurcated needle tip 330 is again advanced into contact with the skin of the recipient. The depth of dermal penetration by bifurcated needle tip 330 for a vaccination can be controlled as desired by selecting a medical device with a different longitudinal distance between the fully extended bifurcated needle tip 330 and the front opening of circular needle guard 310.

Following application of vaccine to a recipient in a dosage and according to a regimen as recommended, compression spring 326 will relax and expand until lip 360 of needle holder 355 abuts inwardly projecting annular rib 362, at which time detachable handle 365 can be disengaged to prevent the subsequent occurrence of accidental needle sticks as might be experienced if detachable handle 365 was not detached and circular thumb cap 364 was advanced again to expose bifurcated needle tip 330 relative to circular needle guard 310. Following detachment of detachable handle 364 from needle holder 355 after a vaccination, the two parts are desirably deposited into a sharps container for safe disposal as previously discussed.

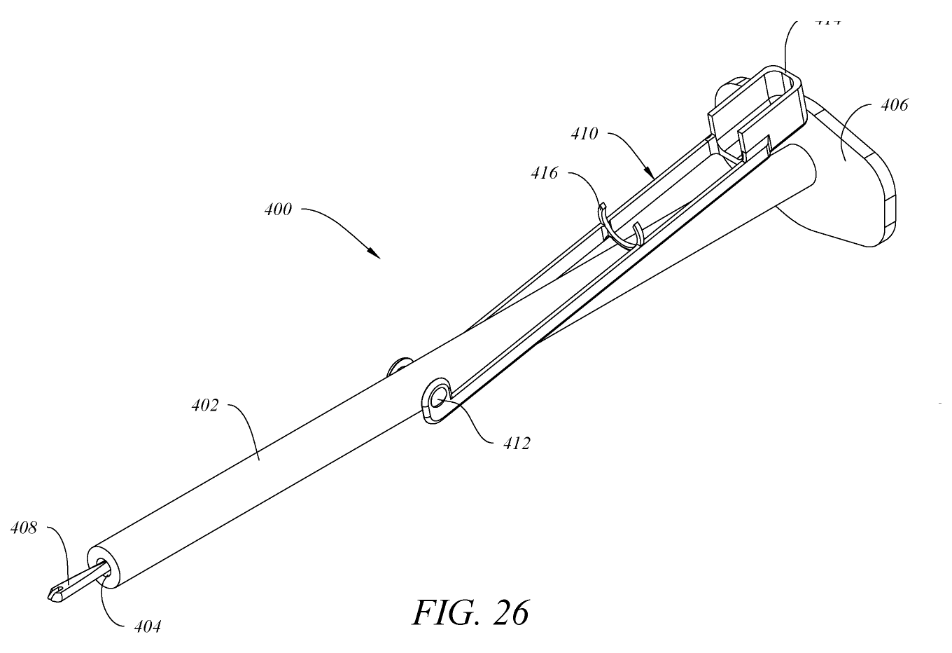

Another medical device 400 useful for safely and effectively administering vaccines or other medicaments to a recipient is disclosed in relation to FIGS. 26-29 of the drawings. Referring to FIGS. 26-28, medical device 400 comprises tubular needle holder 402 with transverse flange 406 at its proximal end and an axial bore 404 of sufficient length to receive and hold needle rod 418 in fixed relation to needle holder 402 with bifurcated needle tip 408 projecting forwardly from the distal end of needle holder 402. The distance that bifurcated needle tip 408 projects forwardly from needle holder 402 is suitable for achieving a desired degree of dermal penetration when applying a vaccine or other medicament to a recipient.

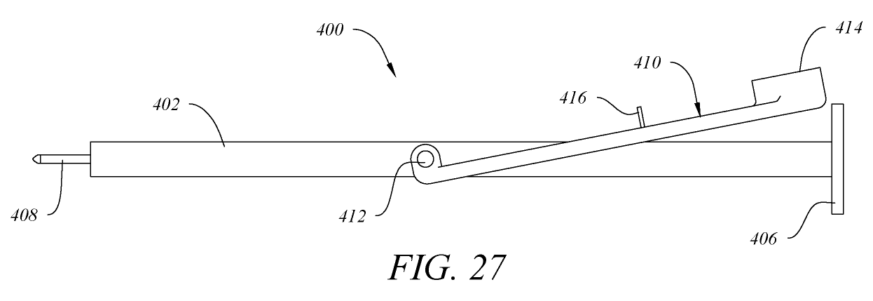

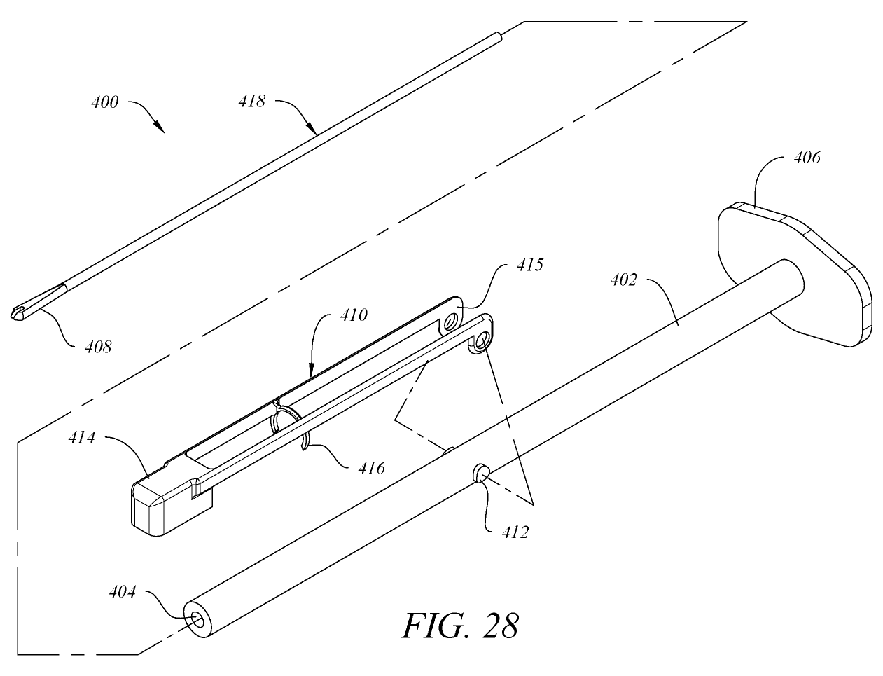

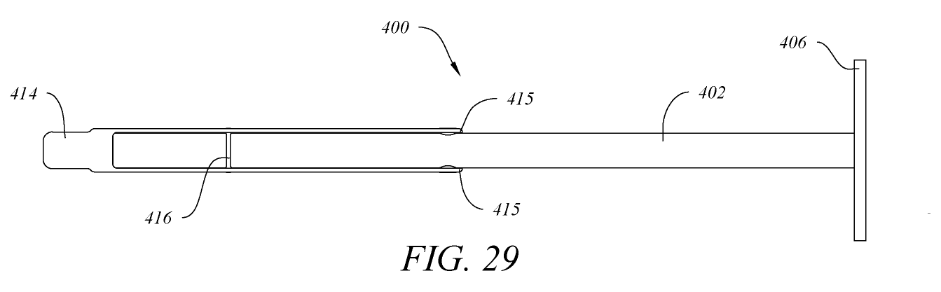

With this embodiment of the invention, a pivotable needle guard 410 is provided that is selectively pivotable between a use position as depicted in FIGS. 26- 27 to a pre-use and post-use position as shown in FIGS. 28-29. Pivotable needle guard 410 further comprises two parallel and spaced-apart elongate arms having rotatable hinge members 415 each pivotably attached to one of two oppositely projecting bosses 412 or other similarly effective structures projecting outwardly on each side of needle holder 402 approximately midway between front opening 404 and transverse finger flange 406. Protective cover 414, disposed at the ends of the elongate arms that are opposite the rotatable hinge members 415, is desirably sized and configured to cover and protect bifurcated needle tip 408 from accidental needle sticks when medical device 400 is not in use. If desired, arcuate support member 416 is provided to connect the oppositely disposed elongate arms to each other to strengthen and provide additional rigidity to pivotable needle guard 410, and can be configured to rest upon, conform to and frictionally hold the tubular body of needle holder 402 when pivoted to the position shown in FIGS. 28-29 when medical device 400 is not in use. When disposed for use in the position shown in FIGS. 26-27, medical device 400 is desirably held by the user similarly to the way a pencil is gripped while inserting bifurcated needle tip 408 into a receptacle containing a vaccine or other medicament and then repeatedly advancing bifurcated needle tip 408 against the skin of a recipient to achieve a desired degree of dermal penetration while transferring a full dose of the vaccine to the recipient. Once the vaccine has been administered, protective needle guard 410 is desirably pivoted again to the position shown in FIGS. 28-29 with protective cover 414 shielding bifurcated needle tip 408 against accidental needle sticks until medical device 400 can be safely deposited in a sharps container for subsequent handling and disposal.

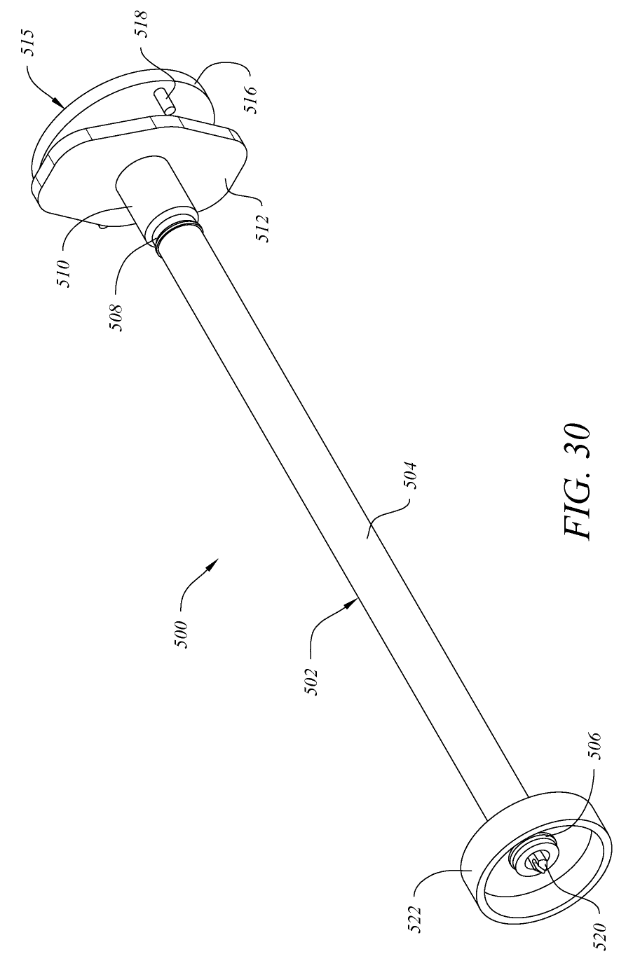

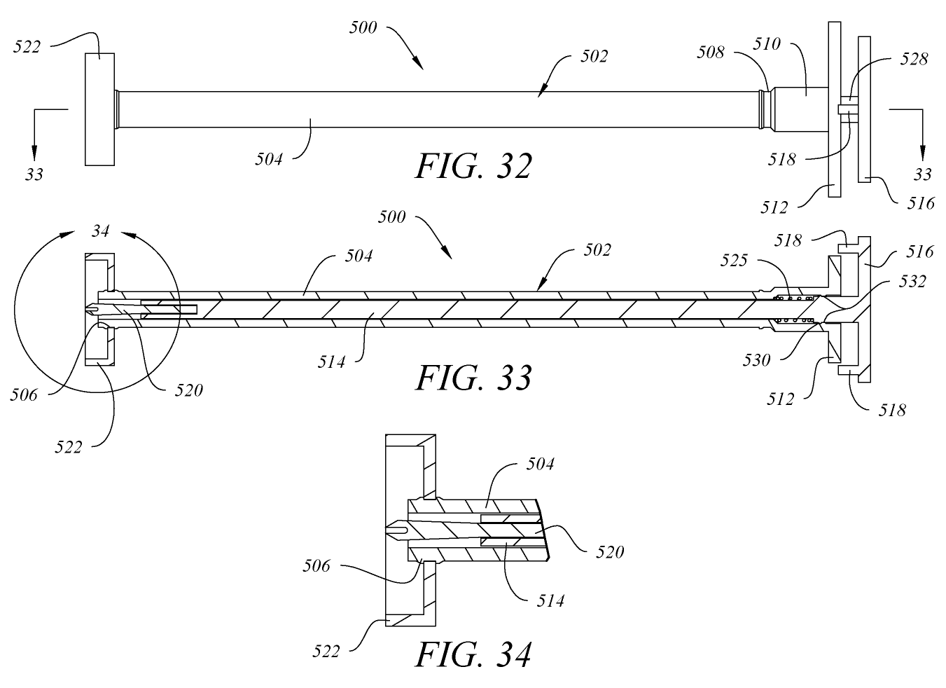

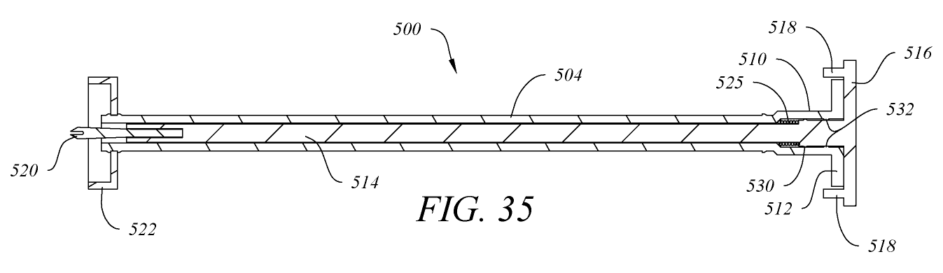

Referring to FIGS. 30-35 of the drawings, another embodiment of the invention is disclosed in relation to medical device 500, which is configured and functions similarly to medical device 300 described above. As disclosed here, medical device 500 further comprises tubular barrel 502 having a front section 504 with a bore sized and configured to receive and support longitudinal shaft 514 of needle holder 515. Circular needle guard 522 desirably comprises a forwardly facing circular opening and a rear wall with a centrally disposed aperture 524 attachable to annular seating structure 506 disposed at the distal end of tubular barrel 502. Tubular barrel 502 is configured to receive the front portion of needle holder 515 through a rearwardly facing opening in transverse finger flange 512, which is attached in fixed relation to annular collar 510 that provides a seating annulus for compression spring 525.

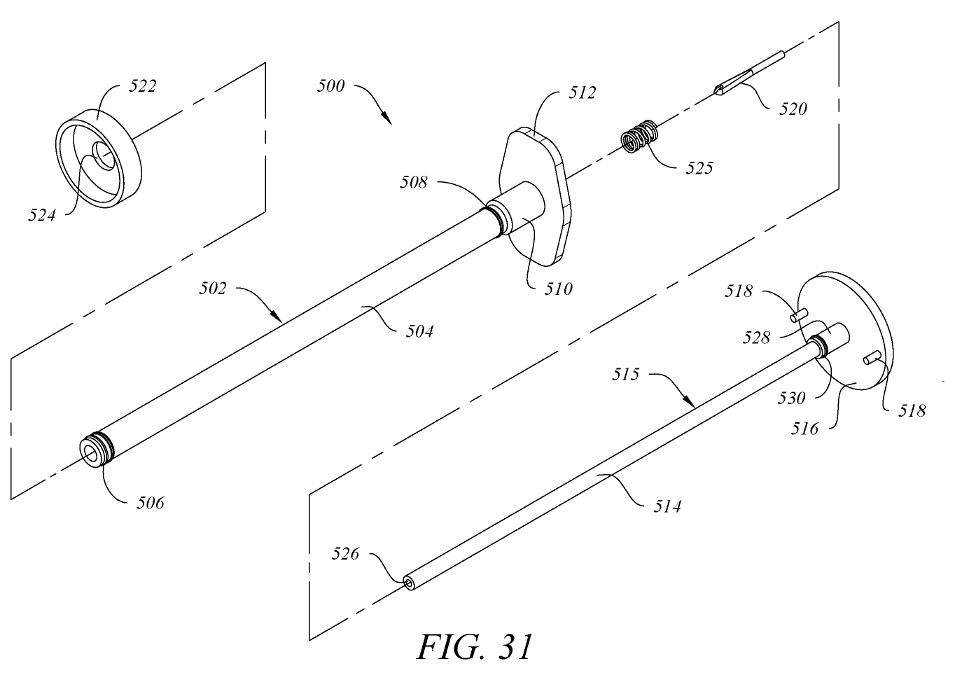

Needle holder 515 comprises proximal end cap 516 attached in fixed relation to shaft section 528 that is insertable into the rear opening of tubular barrel 502, and proximal end cap 516 further comprises two spaced-apart forwardly projecting guideposts 518 that facilitate axial reciprocation of needle holder 515 relative to finger flange 512 of medical device 500. Referring to FIGS. 30, 32-34, bifurcated needle tip 520 is withdrawn inside circular needle guard 522 whenever no forwardly directed force is being applied to the proximal face of end cap 516 and compression spring 525 has biased needle holder 515 rearwardly to a point where outwardly facing annular seal 530 of shaft section 528 frictionally engages inwardly facing annular ridge 532 disposed inside the rear opening into finger flange 512. Referring to FIGS. 31, 35, bifurcated needle tip 520 is advanced forwardly of circular needle guard 522 into contact with the skin of a recipient when a forwardly directed manual force is applied to the proximal face of end cap 516, thereby pressing the distal face of thumb cap 516 into abutting contact with the proximal face of finger flange 512 while compressing compression spring 525. Compressed spring 525 then biases needle holder 515 and bifurcated needle tip 520 rearwardly for a subsequent manual reciprocation of needle holder 515 relative to tubular barrel 502 for multiple strokes (up to about 15) during vaccination. When the vaccination is complete and manual pressure by the user is no longer forcing bifurcated needle tip 520 forwardly out of circular needle guard 522, compression spring 525 relaxes and medical device 500 returns to the safe and protected position of FIG. 35 pending disposal of medical device 500 in a suitable sharps container.

Other alterations and modifications of the invention will likewise become apparent to those of ordinary skill in the art upon reading this specification in view of the accompanying drawings, and it is intended that the scope of the invention disclosed herein be limited only by the broadest interpretation of the appended claims to which the inventors are legally entitled.

1. A medical device, comprising:
a needle comprising a needle shaft and a bifurcated needle tip disposed at a distal end of the needle shaft;
a needle holder comprising a coaxial bore configured to receive a proximal end of the needle shaft, wherein the bifurcated needle tip projects forwardly from the coaxial bore; and
a selectively positionable safety device slidable along the needle holder to guard the bifurcated needle tip in a first stationary “non-use” position and expose the bifurcated needle tip in a second stationary “use” position.

2. The medical device of claim 1, wherein the needle holder is slidably received through a cylindrical bore formed in a cylindrical body of the safety device.

3. The medical device of claim 2, wherein the needle holder comprises a first pair of annular shoulders disposed at a distal end of the needle holder and a second pair of annular shoulders disposed rearwardly from the first pair of annular shoulders.

4. The medical device of claim 3, wherein a first annular seating groove is formed between the first pair of annular shoulders, and wherein a second annular seating groove is formed between the second pair of annular shoulders.

5. The medical device of claim 4, wherein the safety device comprises an inwardly facing annular rib configured to engage the first annular seating groove in the first stationary “non-use” position and engage the second annular seating groove in the second stationary “use” position.

6. The medical device of claim 5, wherein the inwardly facing annular rib is disposed about the cylindrical bore near a proximal end of the safety device.

7. The medical device of claim 1, wherein the coaxial bore of the needle holder comprises undulations adjacent to a front opening of the coaxial bore for affixing the needle within the coaxial bore of the needle holder.

8. The medical device of claim 1, wherein the safety device comprises a pair of radially projecting wings.

9. The medical device of claim 8, wherein the needle holder comprises a transversely projecting flange disposed at a proximal end of the needle holder.

10. The medical device of claim 9, wherein the wings are configured to aid in repositioning the safety device when rearwardly directed manual pressure is applied to the wings against an opposing manual force applied to the flange of the needle holder .

11. The medical device of claim 1, wherein the bifurcated needle tip is positioned inside a cylindrical bore of the safety device in the first stationary “non-use” position.

12. The medical device of claim 11, wherein a distal end of the safety device is positioned forwardly of the bifurcated needle tip in the first stationary “non-use” position.

13. The medical device of claim 12, wherein the distal end of the safety device is positioned rearwardly of the bifurcated needle tip in the second stationary “use” position to expose the bifurcated needle tip for administration of a dose of a vaccine or other medicament.

14. The medical device of claim 13, wherein the medical device is useful for administering the dose of the vaccine or other medicament to a recipient by repeatedly making intradermal contact to a selected area of skin of the recipient with the bifurcated needle tip.

15. A medical device useful, comprising:
a needle comprising a needle shaft and a bifurcated needle tip projecting forwardly from a distal end of the needle shaft;
a needle holder comprising a coaxial bore configured to receive a proximal end of the needle shaft and secure the needle in fixed relation to the coaxial bore; and
a selectively positionable safety device slidable along the needle holder between a first stationary position where the safety device surrounds the bifurcated needle tip to guard the bifurcated needle tip against accidental contact and a second stationary position where the bifurcated needle tip is exposed for use of the medical device.

16. The medical device of claim 15, wherein the needle holder comprises a first annular seating groove disposed between a first set of outwardly facing annular shoulders disposed at a distal end of the needle holder, and wherein the safety device comprises an inwardly facing annular rib disposed about a cylindrical bore of the safety device and configured to frictionally engage the first annular seating groove to retain the safety device in the first stationary position.

17. The medical device of claim 16, wherein the needle holder comprises a second annular seating groove disposed between a second set of outwardly facing annular shoulders disposed rearwardly from the first set of outwardly facing annular shoulders along the needle holder, and wherein the inwardly facing annular rib is configured to frictionally engage the second annular groove to retain the safety device in the second stationary position.

18. The medical device of claim 17, wherein the cylindrical bore of the safety device comprises an inside diameter greater than an outside diameter of each of the first set of outwardly facing annular shoulders and the second set of annular shoulders to allow the safety device to move past the annular shoulders and selectively slide therebetween.

19. A method of operating a medical device, comprising:
providing the medical device comprising: a needle comprising a needle shaft and a bifurcated needle tip projecting forwardly from a distal end of the needle shaft; a needle holder comprising a coaxial bore configured to receive a proximal end of the needle shaft and secure the needle in fixed relation to the coaxial bore; and a selectively positionable safety device slidable along the needle holder between a first stationary position where the safety device surrounds the bifurcated needle tip to guard the bifurcated needle tip against accidental contact and a second stationary position where the bifurcated needle tip is exposed for use of the medical device;
sliding the safety device from the first position where the safety device engages a first annular seating groove on the needle holder and the safety device surrounds the bifurcated needle tip to guard the bifurcated needle tip against accidental contact to the second position where the safety device engages a second annular seating groove and the bifurcated needle tip is exposed for use of the medical device; and
administering a dose of a vaccine or other medicament to a recipient by making one or more intradermal contacts to a selected area of skin of a recipient with the bifurcated needle tip when the safety device is in the second stationary position.

20. The method of claim 19, further comprising:
sliding the safety device from the second stationary position to the first stationary position following the making of a final intradermal contact of the one or more intradermal contacts to protect a user from inadvertent contact with the bifurcated needle tip.
